(19) United States
(12) Patent Application Publication
(10) Pub. No.: US 2022/0080019 A1
(43) Pub. Date: Mar. 17, 2022

(54) ORAL ARTICLES AND METHODS OF USE

(71) Applicant: 3M INNOVATIVE PROPERTIES COMPANY, St. Paul, MN (US)

(72) Inventors: Hannah C. Cohen, St. Paul, MN (US); Katie F. Wlaschin, St. Paul, MN (US); Yizhong Wang, Woodbury, MN (US); Amanda C. Engler, Woodbury, MN (US); Jie Yang, Woodbury, MN (US); Tiffany T. Ton, Woodbury, MN (US); Joel D. Oxman, Minneapolis, MN (US)

(21) Appl. No.: 17/419,516

(22) PCT Filed: Dec. 26, 2019
(86) PCT No.: PCT/IB2019/061378

§ 371 Date: Jun. 29, 2021

Related U.S. Application Data
(60) Provisional application No. 62/786,357, filed on Dec. 29, 2018.

(51) Int. Cl. A61K 36/886; A61K 9/00; A61K 47/44; A61K 47/42; A61K 47/36; A61K 31/519; A61K 31/728; A61K 31/198; A61K 31/205; A61K 31/355; A61K 31/714; A61K 31/4425; A61K 31/155; A61K 45/06; A61K 47/02

(52) U.S. Cl. CPC A61K 36/886; A61K 47/02; A61K 47/44; A61K 47/42; A61K 47/36; A61K 31/519; A61K 31/728; A61K 31/198; A61K 31/205; A61K 31/355; A61K 31/714; A61K 31/4425; A61K 31/155; A61K 45/06; A61K 9/0056

(57) ABSTRACT

Articles that include not greater than 60 wt-% of one or more plant based oil that is solid at 25° C. (room temperature), the weight percent based on the total weight of the article; and from 4 wt-% to 10 wt-% of gelatin, from 0.8 to 5 wt-% of agar, or a combination thereof, the weight percent based on the total weight of the article, wherein the article is solid set and homogeneous. Methods of using the articles and compositions for forming the articles are also disclosed.

### SUMMARY

Disclosed herein are articles that include not greater than 60 wt-% of one or more plant based oil that is solid at 25° C. (room temperature), the weight percent based on the total weight of the article; and from 4 wt.-% to 10 wt.-% of gelatin, from 0.8 to 5 wt.-% of agar, or a combination thereof, the weight percent based on the total weight of the article, wherein the article is solid set and homogeneous.

Also disclosed are methods of preventing, inhibiting, disrupting, or any combination thereof the formation or maintenance of a biofilm in an oral tissue, the method including contacting an oral tissue with a disclosed article(s).

Also disclosed are method of affecting hydration loss in an oral tissue, the method including contacting an oral tissue with a disclosed article(s).

Also disclosed are methods of affecting lubricity or lubriciousness in an oral tissue, the method including contacting an oral tissue with a disclosed article(s).

Also disclosed are methods of affecting the effects of xerostomia, dry mouth, or both, the method including contacting an oral tissue with a disclosed article(s).

Also disclosed are compositions for forming an article, the composition including not greater than 50 wt-% of one or more plant based oil that is solid at 25° C. (room temperature), the weight percent based on the total weight of the composition; from 2 wt-% to 15 wt-% of gelatin, from 0.6 to 4.8 wt-% of agar, or a combination thereof, the weight percent based on the total weight of the composition; and from 20 wt-% to 60 wt-% water based on the total weight of the composition.

The above summary is not intended to describe each embodiment of the present disclosure. The details of one or more embodiments of the present disclosure are also set forth in the description below. Other features, objects, and advantages of the present disclosure will be apparent from the description and from the claims.

### DETAILED DESCRIPTION

Xerostomia or dry mouth is a common condition that results from insufficient saliva volume. It is increasingly prevalent in the aging population and is a side-effect of many medications, as well as cancer treatment. Severe cases of xerostomia are often related to salivary gland dysfunction, known as Sjögren's Syndrome.

The lack of moisture and lubrication typically provided by saliva has a range of negative effects on oral tissue (soft tissue) ranging from mild discomfort to extremely painful and infected mouth sores. The persistent discomfort and dryness can also contribute to larger health issues by causing disruption of sleep, and impairing one's ability to talk (socialize, may impact psychological health) and eat (may impact nutrition). Dry buccal tissue is a less effective barrier and more susceptible to penetration by physical irritants such as toxins and carcinogens in foods, beverages and tobacco.

Saliva is also the mouth's primary defense against tooth decay. Healthy saliva flow helps prevent cavities by physically removing bacteria from the oral cavity before they can become attached to tooth and tissue surfaces and form a protected biofilm. The flow of saliva also helps dilute sugars and acids introduced by intake of food and beverages. The buffering capacity neutralizes acids and aids in the digestive process. The presence of calcium and phosphate salts provides continuous opportunity for re-mineralization of tooth enamel, serving to reverse the tooth decay process.

Many who suffer with xerostomia use separate products to address hard tissue health and soft tissue comfort. For soft tissue comfort, saliva substitute products are typically designed to provide lubrication and moisture. The format of these products is varied, and includes viscous gels/pastes, sprays, rinses, mints, and slow-release tablets. These are applied multiple-times per day or as needed for comfort. For hard-tissue health, different treatments are used to directly address cavity prevention (antiseptic rinses, fluoride products, calcium/phosphate treatments). Often “dry mouth-friendly” versions of products, such as toothpastes and mouth rinses are recommended. Dry mouth friendly products typically have a neutral pH and do not contain alcohol or other irritating components (e.g. anionic surfactants or anionic emulsifiers).

There is a desire to design a single product that effectively and easily addresses the need for dry mouth symptom relief (soft tissue comfort) and oral health preventative benefits (tooth enamel and cavity protection). A fully ingestible, lozenge type product is well suited for this purpose. It addresses both the health of hard-tissue and soft tissue.

Disclosed herein are articles that can be utilized as oral articles, for example. Disclosed articles can be in the form of a lozenge, for example. Disclosed articles are not gums. A gum does not dissolve in the mouth of a user, whereas a lozenge does dissolve in the mouth of a user.

Disclosed articles, one or more components in an article, or both can be characterized as edible. Referring to a component, composition or article as edible can mean that the particular ingredient, composition or article is safe for daily, long-term ingestion at recommended use levels. In some embodiments, the GRAS (generally regarded as safe) list from the United States Food and Drug Administration (FDA) can be utilized to determine if a component is edible at the levels utilized in a composition. Disclosed articles include one or more useful oils that can be a solid(s) at room temperature and gelatin, agar, or combinations thereof. Disclosed articles can also be described by compositions that can be used to prepare disclosed oral articles. Disclosed articles that can be useful by a user can be described as solid set and homogeneous. “Solid set” as used herein means that the article does not flow at 25° C. (room temperature).

Disclosed articles include one or more oil that is a solid at room temperature (25° C.). In some embodiments, useful oils do not include silicone based oils (e.g., simethicone or dimethicone). Useful oils can include any oils but in some embodiments can include plant based oils. Many plant based oils can be made to exist as a solid at room temperature by hydrogenating them. The articles can include a single edible oil, or as many as two, three, four, five or more edible oils. Examples of suitable edible oils that are solid at room temperature can include, but are not limited to hydrogenated plant based oils and the like, and mixtures or fractions thereof. A specific example includes cocoa butter.

Disclosed articles can include not greater than 60 percent of one or more oils that are solid at 25° C. (room temperature) based on the total weight of the article, not greater than 57 percent of one or more oils that are solid at room temperature based on the total weight of the article, not greater than 56 percent of one or more oils that are solid at room temperature based on the total weight of the article, not greater than 52 percent of one or more oils that are solid at room temperature based on the total weight of the article, or not greater than 50 percent of one or more oils that are solid at room temperature based on the total weight of the article. Disclosed articles can include not less than 5 percent of one or more oils that are solid at room temperature based on the total weight of the article, not less than 10 percent of one or more oils that are solid at room temperature based on the total weight of the article, not less than 15 percent of one or more oils that are solid at room temperature based on the total weight of the article, or not less than 19 percent of one or more oils that are solid at room temperature based on the total weight of the article.

Compositions for forming disclosed useful articles can include not greater than 50 percent of one or more oils that are solid at 25° C. (room temperature) based on the total weight of the composition, not greater than 48 percent of one or more oils that are solid at room temperature based on the total weight of the composition, not greater than 45 percent of one or more oils that are solid at room temperature based on the total weight of the composition, or not greater than 40 percent of one or more oils that are solid at room temperature based on the total weight of the composition. Compositions for forming disclosed articles can include not less than 5 percent of one or more oils that are solid at room temperature based on the total weight of the composition, not less than 10 percent of one or more oils that are solid at room temperature based on the total weight of the composition, not less than 15 percent of one or more oils that are solid at room temperature based on the total weight of the composition, or not less than 19 percent of one or more oils that are solid at room temperature based on the total weight of the composition.

Disclosed articles and compositions for forming disclosed articles can also include gelatin, agar, or combinations thereof.

In some embodiments, disclosed articles can include not less than 4 percent gelatin based on the total weight of the article, not less than 5 percent gelatin based on the total weight of the article, or not less than 6 percent gelatin based on the total weight of the article. In some embodiments, disclosed articles can include not greater than 10 percent gelatin based on the total weight of the article, not greater than 9 percent gelatin based on the total weight of the article, or not greater than 8 percent gelatin silica based on the total weight of the article.

Compositions for forming disclosed useful articles can include not less than 2 percent gelatin based on the total weight of the article, not less than 4 percent gelatin based on the total weight of the article, not less than 5 percent gelatin based on the total weight of the article, or not less than 6 percent gelatin based on the total weight of the article. In some embodiments, disclosed articles can include not greater than 15 percent gelatin based on the total weight of the article, disclosed articles can include not greater than 10 percent gelatin based on the total weight of the article, not greater than 8 percent gelatin based on the total weight of the article, or not greater than 7 percent gelatin based on the total weight of the article.

In some embodiments, disclosed articles can include not less than, not less than 0.8 percent agar based on the total weight of the article, not less than 1 percent agar based on the total weight of the article, not less than 1.2 percent agar based on the total weight of the article, or not less than 1.6 percent agar based on the total weight of the article. In some embodiments, disclosed articles can include not greater than 5 percent agar based on the total weight of the article, not greater than 4 percent agar based on the total weight of the article, or not greater than 2 percent agar based on the total weight of the article.

Compositions for forming disclosed useful articles can include not less than 0.6 percent agar based on the total weight of the article, not less than 0.8 percent agar based on the total weight of the article, not less than 1 percent agar based on the total weight of the article, or not less than 1.4 percent agar based on the total weight of the article. In some embodiments, disclosed articles can include not greater than 4.8 percent agar based on the total weight of the article, not greater than 3.8 percent agar based on the total weight of the article, or not greater than 1.8 percent agar based on the total weight of the article.

In some embodiments, compositions for making disclosed articles and disclosed useful articles can also optionally include water. The water can be used to contain water soluble materials within the final article and/or aid in processing and manufacturing of disclosed articles. In some embodiments, water can be added to a composition to make a disclosed article in combination with a number of different components, for example as a buffer solution. Illustrative materials can include minerals (e.g., calcium), sweeteners, etc. In some embodiments, compositions for making disclosed articles can include not less than 20 percent water based on the total weight of the composition, or not less than 25 percent water based on the total weight of the composition. In some embodiments, compositions for forming disclosed articles can include not greater than 60 percent water based on the total weight of the composition, or not greater than 57 percent water based on the total weight of the composition. In some embodiments, disclosed articles can include not less than 10 percent water based on the total weight of the article, or not less than 12 percent water based on the total weight of the article. In some embodiments, disclosed articles can include not greater than 35 percent water based on the total weight of the article, or not greater than 33 percent water based on the total weight of the article.

Disclosed articles and/or compositions for making such articles can also optionally include additional components other than those discussed above. Illustrative optional components can include, for example, sweeteners (e.g., non-carcinogenic sweeteners), mineral salts, buffering components, flavorants, preservative agents, humectants, or combinations thereof. Other optional beneficial ingredients can also be included at appropriate levels such as, aloe vera (multi-benefit), folic acid (related to B12), hyaluronic acid (lubricating, moisturizing), ceramides , amino acids (e.g., glycine, arginine), betaines or oxygenated glycerol triesters, vitamin E (antioxidant), vitamin B12 EDTA, cetyl pyridinium chloride, chlorhexidine, other antiseptics , etc., or combinations thereof.

In some embodiments, disclosed articles and/or compositions for forming such articles can include flavorants including for example, spearmint, peppermint, strawberry, butter, vanilla, coconut, almond, bubble gum, berry, fruit punch, butterscotch, caramel, or combinations thereof. In some embodiments, some flavorants, e.g., mint, citrus, etc. can also be advantageous because they stimulate salivary production when utilized in articles. Artificial sweeteners may also be used (stevia, aspartame, sucralose, neotame, acesulfame potassium (Ace-K), saccharin, and advantame, for example). In some embodiments, disclosed articles can include one or more sweeteners including for example, non-cariogenic polyols, or sugar substitutes (e.g., sucralose). In some embodiments, disclosed articles can include non-cariogenic polyol sweeteners such as xylitol, sorbitol, maltitol, erythritol, isomalt, or combinations thereof. In some embodiments, disclosed articles can include non-cariogenic polyol sweeteners such as xylitol, sorbitol, or combinations thereof. In articles that include optional sweeteners, the sweetener can be present in an amount that is not less than 2.5 percent based on the total weight of the article or not less than 1 percent based on the total weight of the article. In some embodiments, an optional sweetener can be present in an amount that is not greater than 30 percent based on the total weight of the article, not greater than 15 percent based on the total weight of the article, or not greater than 8 percent based on the total weight of the article. In some embodiments, compositions for forming disclosed articles can contain amounts of sweeteners that range from 10 percent to 30 percent based on the weight of the total composition, for example.

In some embodiments, disclosed articles and/or compositions for forming such can optionally include one or more minerals that may be useful or beneficial for ingestion or oral health. Illustrative optional minerals that can be included in disclosed articles can include calcium (Ca), phosphorus (P), magnesium (Mg), fluorine (F), iron (Fe), strontium (Sr), zinc (Zn), potassium (K), or combinations thereof. In some embodiments, some minerals can be provided by including magnesium chloride (MgCl₂), calcium chloride (CaCl₂), strontium chloride, zinc chloride, zinc gluconate, potassium nitrate, potassium phosphate dibasic (KH₂PO₄), or combinations thereof In some embodiments, where fluorine is included, it can be included as the fluoride ion (F—) in salt form (MgF₂, CaF₂, etc.), at a concentration that is not greater than 4 milligrams per liter (mg/L).

In some embodiments, disclosed articles and/or compositions for forming such can include one or more preservatives to render the article microbiologically stable, to increase the microbiological stability thereof, or some combination thereof. In some embodiments, useful preservatives include those that work at a neutral pH, do not detrimentally affect taste, are edible, are effective against a broad spectrum of pathogens, or combinations thereof. Specific illustrative useful preservatives can include GEOGARD® preservatives, which are commercially available from Lonza (Basel, Switzerland) and include salicyclic acid, benzyl alcohol, sodium benzoate, potassium sorbate, parabens, natural preservatives, polyglyceryl esters, monolaurin,1,2 octanediol, caprylic/capric triglycerides, DHA, aloe vera, potassium sorbate, cetyl pyridinium chloride (CPP), polyhexamethylene biguanide (PHMB), methylparaben, and chlorhexidine gluconate (CHG) for example.

Disclosed articles can generally be formed by dissolving the gelatin, agar or combination thereof and any optional other water soluble components in water or optionally a buffer solution containing water and optional minerals to form a water phase. The mixture may be heated in order to facilitate dissolving the various components. The oil phase can be prepared by heating the one or more oil that is a solid at 25° C. (room temperature) to melting and then adding optional ingredients (e.g., optional thickeners such as ethylcellulose, fumed silica etc.), emulsifiers, etc. The water based composition and the oil based composition can then be mixed together to form a composition capable of forming a disclosed article. The mixture can be further mixed, cooled, etc. In some embodiments, the mixture can be homogenized after mixing the water phase and the oil phase together.

Disclosed articles can have varied properties. In some embodiments, disclosed articles can be described by the pH thereof, the viscosity thereof, the stability thereof, various other properties, or combinations thereof.

In some embodiments, disclosed articles can have a pH that is acceptable for use in the mouth of a person, for example. In some embodiments, disclosed articles can have a pH from 4.5 to 9.5, for example. In some embodiments, the article can have a pH in a more neutral range from 5.0-8.5 or 5.5-8.5 for example, as dry mouth sufferers can have a higher sensitivity to pH. The article can naturally have such a pH or can be buffered to have a pH in a useful, e.g., a “neutral” range.

In some embodiments, disclosed articles can be described as solid set (e.g., the article does not flow and retains its shape) and homogenous (e.g., the article has no visible separation). In some embodiments, disclosed articles can be described as solid set and homogenous even after re-heating, cooling, or any combinations thereof. Articles that are solid set and homogeneous even after any of re-heating, cooling, or any combination thereof may be advantageous due to extreme environmental conditions that they may be subjected to by a user (e.g., storage of the articles in automobiles, which could subject them to extreme temperatures). Disclosed useful articles can have amounts of water varying from 10 percent to 35 percent water based on the total weight of the article, or from 12 to 34 percent water based on the total weight of the article.

Disclosed articles can be packaged in any of a number of commonly utilized fashions, including for example blister packages, bags, etc. The articles themselves can also be molded into virtually any size or shapes.

In some embodiments, disclosed articles can have desired effects when utilized. Such effects can include, for example the article's effect on biofilms, the article's effect on plaque buildup, the article's effect on water loss, the article's ability to maintain or provide lubricating properties, resist dilution or wash-off by saliva or water, or drinking and eating in general or combinations thereof.

In some embodiments, disclosed articles can prevent, inhibit, disrupt the formation or maintenance of a biofilm in an area contacted with the article. The area contacted can be in vivo or in vitro. In some embodiments, an article can prevent, inhibit, disrupt the formation or maintenance of a biofilm in a mouth of a user where the article was applied to the mouth, for example via placing the article into the mouth when compared to a mouth without the article applied thereto. In some embodiments, an article can prevent, inhibit, disrupt the formation or maintenance of a biofilm in a container in which a biofilm exists and the article was applied to the container via contact when compared to a container without the article contacted thereto. Preventing, inhibiting, disrupting, or some combination thereof the formation or maintenance of biofilms can be measured using a modified version of the MBEC assay (described in ASTM E2799), which measures disruption of Strep mutans biofilms grown on special pegs in a microtiter plate. The biofilms growing on the pegs are treated by periodic submersion into test materials, followed by washing in saliva and water. The biofilm remaining on each peg following treatment is quantified by measuring the amount of fluorescently labeled bacteria that eluted from the pegs at the end of the treatment cycles (see example). In some embodiments, disclosed articles can affect the buildup of plaque in an area contacted by the article. The area contacted can be in vivo or in vitro. In some embodiments, an article can decrease plaque buildup on at least one tooth in a mouth of a user where the article was applied to the mouth, for example via contacting the article into the mouth when compared to a mouth without the article applied thereto. In some embodiments, an article can decrease plaque buildup in a container in which plaque can develop and the article was applied to the container via pouring, spraying, etc. when compared to a container without the article contacted thereto. Decreasing plaque buildup can be measured by a variety of methods in vivo including for example plaque scoring, dyeing of plaque, etc.

In some embodiments, disclosed articles can affect hydration loss in an area contacted by the articles. The area contacted can be in vivo or in vitro. In some embodiments, an article can decrease hydration loss in a mouth of a user where the article was applied to the mouth, for example via contacting the article into the mouth when compared to a mouth without the article applied thereto. In some embodiments, an article can decrease hydration loss from a tissue in which hydration can be lost and the article was applied to the tissue via contact when compared to a tissue without the article applied thereto.

In some embodiments, disclosed articles can affect lubricity or lubriciousness of an area contacted by the article. The area contacted can be in vivo or in vitro. In some embodiments, an article can maintain or increase lubricity in a mouth of a user where the article was applied to the mouth, for example via contacting the article into the mouth when compared to a mouth without the article applied thereto.

Also disclosed herein are methods of using disclosed articles. Disclosed methods can include contacting an oral cavity or oral tissue with a disclosed article. The step of contacting the oral cavity or oral tissue can be accomplished by applying the article in any way, for example by simply placing the article in the mouth. Disclosed methods can be useful for preventing, inhibiting, disrupting, or any combination thereof the formation or maintenance of a biofilm in an area contacted with the article; for affecting hydration loss in an area contacted by the article; for affecting lubricity or lubriciousness of an area contacted by the article; for affecting or alleviating the effects of xerostomia, dry mouth, or both.

All scientific and technical terms used herein have meanings commonly used in the art unless otherwise specified. The definitions provided herein are to facilitate to understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. For example, a composition that “comprises” silver may be a composition that “consists of” silver or that “consists essentially of” silver.

As used herein, “consisting essentially of,” as it relates to a composition, apparatus, system, method or the like, means that the components of the composition, apparatus, system, method or the like are limited to the enumerated components and any other components that do not materially affect the basic and novel characteristic(s) of the composition, apparatus, system, method or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Also herein, the recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” a particular value, that value is included within the range.

Use of “first,” “second,” etc. in the description above and the claims that follow is not intended to necessarily indicate that the enumerated number of objects is present. For example, a “second” substrate is merely intended to differentiate from another substrate (such as a “first” substrate). Use of “first,” “second,” etc. in the description above and the claims that follow is also not necessarily intended to indicate that one comes earlier in time than the other.

Example articles and techniques according to the disclosure provide will be illustrated by the following non-limiting examples.

### EXAMPLES

TABLE 1
Materials

| Chemical/Material Name | Abbreviation | CAS | Manufacturer |
|---|---|---|---|
| Dulbecco's phosphate buffered | DPBS | | Gibco (Life |
| saline (contains 100 mg/L CaCl₂, | | | Technologies |
| 100 mg/L MgCl₂—6H₂O, 200 mg/L | | | Limited), Paisley, |
| KCl, 200 mg/L KH₂PO₄, 8 g/L | | | UK |
| NaCl, 2.16 g/L Na₂HPO₄—7H₂O | | | |
| (DPBS) | | | |
| Knox Original Unflavored Gelatine | Gelatin | | E.D. Smith Foods, |
| | | | Ltd, Ontario, Canada |
| Agar-Agar Powder | Agar | | Living Jin Mannam- |
| | | | ro, Dongnam-gu, |
| | | | Cheonan-si, |
| | | | Republic of Korea |
| Acacia Gum (Gum Arabic from | | | Sigma Aldrich, St. |
| Acacia Tree) | | | Louis MO |
| WECOBEE M (solid at 25° C., room | | | Stepan Lipid |
| temperature) | | | Nutrition, Maywood, |
| | | | NJ |
| Xylitol | | | Now Foods, |
| | | | Bloomingdale, IL |
| Glycerol | Glycerin | | VWR International, |
| | | | LLC Radnor, PA |
| NEOBEE M5 Oil | | | Stepan, Maywood, |
| | | | NJ |
| Polyglyceryl-10 Stearate | POLYALDO | 79777-30-3 | Lonza, Basel, |
| (POLYALDO 10-1-S) | 10-1-S | | Switzerland |
| POLYALDO HGDS KFG | 6-2-S | Emulsifier | Lonza Consumer |
| | | | Care, South |
| | | | Plainfield, NJ |
| POLYALDO 10-2-P KFG | 10-2-P | Emulsifier | Lonza Consumer |
| | | | Care, South |
| | | | Plainfield, NJ |
| Aqualon ™ EC-N50 PHARM ethyl | | | Ashland, |
| cellulose | | | Wilmington, DE |
| Locus (Carob) Bean gum | | Hydrocolloid | Spectrum Chemical, |
| | | | Gardena, CA |

### Method of Making Lozenges

The water phase was made by dissolving the hydrocolloid (gelatin, agar, etc.), sugar alcohol and optionally, water-soluble emulsifiers (e.g., soy lecithin, 10-1-S) into DPBS buffer (water containing calcium and phosphate, etc.) and heating along with stirring to the melt temperature of the hydrocolloid (gelatin: ˜45° C., agar: ˜85° C.) until fully mixed. The wt % in the tables for the CaCl₂, MgCl₂-6H₂O, KCl, KH₂PO₄, NaCl, Na₂HPO₄-7H₂O, and H₂O are presented calculated based on the known amount of DPBS buffer added and the amounts of the components therein. The oil phase was prepared by dissolving optional thickeners (e.g., ethylcellulose, fumed silica) and/or emulsifiers (e.g., 6-2-S, 10-2-P) into the WECOBEE M oil at a temperature of ˜80-90° C. with stirring until the mixture was fully dissolved. The oil and water phases were mixed together at varying ratios and cooled to 45° C. They were then homogenized at speed four for two minutes (using a Fisher Scientific PowerGen 1000 Homogenizer). Following homogenization, samples were returned a temperature of 45° C. for 5-10 minutes to observe if separation occurred. Examples that showed no separation were recorded as homogenous. Samples were then poured into silicone molds and set at room temperature. Samples were unmolded once set and then weighed over the course of a few days to monitor weight loss due to water evaporation. Water loss may be accelerated by lyophilization (not exemplified), for example. Percentages listed below are the weight percentages of ingredients added prior to water evaporation, unless otherwise specified.

### Examples 1-3 and Comparative Examples C1 and C2

Example lozenges 1, 2 and 3 as well as comparative examples C1, C2 and C3 were made according to Table 2 below.

TABLE 2
all amounts presented in weight percent (wt-%)

| | | | | Comp. Ex. | Comp. Ex. | Comp. Ex. |
|---|---|---|---|---|---|---|
| | Ex. 1 | Ex. 2 | Ex. 3 | C1 | C2 | C3 |
| Water Phase | | | | | | |
| CaCl₂ | 0.0034 | 0.0030 | 0.0025 | 0.0021 | 0.0017 | 0.0012 |
| MgCl₂—6H₂O | 0.0034 | 0.00230 | 0.0025 | 0.0021 | 0.0017 | 0.0013 |
| KCl | 0.0067 | 0.0059 | 0.00501 | 0.0042 | 0.0034 | 0.0025 |
| KH₂PO₄ | 0.0067 | 0.0059 | 0.0051 | 0.0042 | 0.0034 | 0.0025 |
| NaCl | 0.30 | 0.24 | 0.20 | 0.17 | 0.14 | 0.10 |
| Na₂HPO₄—7H₂O | 0.073 | 0.064 | 0.055 | 0.046 | 0.037 | 0.027 |
| H₂O | 33.3 | 29.2 | 25.0 | 20.9 | 16.7 | 12.6 |
| Xylitol | 27.5 | 24.0 | 20.6 | 17.2 | 13.7 | 10.3 |
| Gelatin | 5.5 | 4.8 | 4.1 | 3.4 | 2.7 | 2.1 |
| 10-1-S | 2.4 | 3.0 | 3.6 | 4.1 | 4.7 | 5.3 |
| Oil Phase | | | | | | |
| Wecobee M | 29.6 | 37.0 | 44.5 | 51.9 | 59.3 | 66.7 |
| Ethylcellulose | 0.7 | 0.9 | 1.1 | 1.3 | 1.5 | 1.7 |
| 6-2-S | 0.6 | 0.8 | 0.9 | 1.1 | 1.2 | 1.4 |
| Homogenous? | Yes | Yes | Yes | No | No | No |

Weights were not recorded on the final lozenges of Examples 1-3 and Comparative Examples C1-C3 so the final weight percentages after water loss (e.g., in the useful article) cannot be calculated.

### Examples 4-7 and Comparative Examples C4 and C5

Example lozenges 4-7 as well as comparative examples C4 and C5 were made according to Table 3 below.

TABLE 3
all amounts presented in weight percent (wt-%)

| | | | | | Comp. Ex. | Comp. Ex. |
|---|---|---|---|---|---|---|
| | Ex. 4 | Ex. 5 | Ex. 6 | Ex. 7 | C4 | C5 |
| Water Phase | | | | | | |
| CaCl₂ | 0.0057 | 0.0050 | 0.0043 | 0.0036 | 0.0028 | 0.0021 |
| MgCl₂—6H₂O | 0.0057 | 0.0050 | 0.0043 | 0.0036 | 0.0028 | 0.0021 |
| KCl | 0.011 | 0.010 | 0.0086 | 0.0072 | 0.0056 | 0.0042 |
| KH₂PO₄ | 0.011 | 0.010 | 0.0086 | 0.0072 | 0.0056 | 0.0042 |
| NaCl | 0.46 | 0.40 | 0.34 | 0.29 | 0.22 | 0.17 |
| Na₂HPO₄—7H₂O | 0.12 | 0.11 | 0.093 | 0.078 | 0.060 | 0.045 |
| H₂O | 56.4 | 49.5 | 42.5 | 35.6 | 27.7 | 20.8 |
| Sorbitol | 20 | 17 | 15 | 12 | 10 | 7 |
| Agar | 2 | 1.4 | 1.2 | 1.0 | 0.8 | 0.6 |
| Glycerin | 2 | 1.4 | 1.2 | 1.0 | 0.8 | 0.6 |
| Locus (carob) | 0.16 | 0.14 | 0.12 | 0.10 | 0.08 | 0.06 |
| bean gum | | | | | | |
| Oil Phase | | | | | | |
| Wecobee M | 19 | 29 | 38 | 48 | 57 | 67 |
| 10-2-P | 1 | 2 | 2 | 3 | 3 | 4 |
| Homogenous? | Yes | Yes | Yes | Yes | No | No |

Weights were not recorded on the final articles of Examples 4-7 and Comparative Examples C4 and C5 so the final weight percentages after water loss, e.g., in the useful article cannot be calculated.

### Examples 8-10 and Comparative Examples C6 and C7

Example lozenges 8-10 as well as comparative examples C6 and C7 were made according to Table 4 below.

TABLE 4
all amounts presented in weight percent (wt-%)

| | | | | Comp. Ex. | Comp. Ex. |
|---|---|---|---|---|---|
| | Ex. 8 | Ex. 9 | Ex. 10 | C6 | C7 |
| Water Phase | | | | | |
| CaCl₂ | 0.0057 | 0.0050 | 0.0043 | 0.0036 | 0.0029 |
| MgCl₂—6H₂O | 0.0057 | 0.0050 | 0.0043 | 0.0036 | 0.0029 |
| KCl | 0.011 | 0.010 | 0.0086 | 0.0072 | 0.0058 |
| KH₂PO₄ | 0.011 | 0.010 | 0.0086 | 0.0072 | 0.0058 |
| NaCl | 0.46 | 0.40 | 0.34 | 0.29 | 0.23 |
| Na₂HPO₄—7H₂O | 0.12 | 0.11 | 0.093 | 0.078 | 0.063 |
| H₂O | 56.4 | 49.5 | 42.5 | 35.6 | 28.7 |
| Sorbitol | 20 | 18 | 15 | 13 | 10 |
| Agar | 1.6 | 1.4 | 1.2 | 1.0 | 0.8 |
| Glycerin | 0.8 | 0.7 | 0.6 | 0.5 | 0.4 |
| Acacia Gum | 0.2 | 0.1 | 0.1 | 0.1 | 0.1 |
| Oil Phase | | | | | |
| Wecobee M | 20 | 29 | 39 | 49 | 59 |
| Fumed Silica | 0.4 | 0.6 | 0.8 | 1.0 | 1.2 |
| Homogenous? | Yes | Yes | Yes | No | No |

Weights were not recorded on the final articles of Comparative Examples C6 and C7 so the final weight percentages after water loss, e.g., in the useful articles cannot be to calculated. Table 5 however, shows the final weights for Examples 8-10 to show the weight change in the articles.

TABLE 5
all amounts presented in weight percent (wt-%)

| | Ex. 8 | Ex. 9 | Ex. 10 |
|---|---|---|---|
| Initial Lozenge | 4.36 | 4.28 | 3.79 |
| Weight (g) | | | |
| Final Lozenge | 2.67 | 2.83 | 2.79 |
| Weight (g) | | | |
| Water Phase | | | |
| CaCl₂ | 0.0030 | 0.0025 | 0.0023 |
| MgCl₂—6H₂O | 0.0030 | 0.0025 | 0.0023 |
| KCl | 0.0061 | 0.0050 | 0.0045 |
| KH₂PO₄ | 0.0061 | 0.0050 | 0.0045 |
| NaCl | 0.24 | 0.20 | 0.18 |
| Na₂HPO₄—7H₂O | 0.065 | 0.054 | 0.049 |
| H₂O | 30.2 | 24.5 | 22.4 |
| Sorbitol | 1.3 | 1.1 | 0.8 |
| Agar | 2.6 | 2.1 | 1.6 |
| Glycerin | 1.3 | 1.1 | 0.8 |
| Acacia Gum | 0.3 | 0.2 | 0.2 |
| Oil Phase | | | |
| Wecobee M | 32.0 | 44.5 | 53.3 |
| Fumed Silica | 0.7 | 0.9 | 1.1 |
| Homogenous? | Yes | Yes | Yes |

### Comparative Examples C8 and C9 and Examples 11 and 12

Comparative Examples C8 and C9 and Examples 11-12 were made according to Table 6 below.

TABLE 6
all amounts presented in weight percent (wt-%)

| | Comp. Ex. | Comp. Ex. | | |
|---|---|---|---|---|
| | C8 | C9 | Ex. 11 | Ex. 12 |
| Water Phase | | | | |
| CaCl₂ | 0.0019 | 0.0024 | 0.0029 | 0.0034 |
| MgCl₂—6H₂O | 0.0019 | 0.0024 | 0.0029 | 0.0034 |
| KCl | 0.0038 | 0.0048 | 0.0058 | 0.0068 |
| KH₂PO₄ | 0.0038 | 0.0048 | 0.0058 | 0.0068 |
| NaCl | 0.15 | 0.19 | 0.23 | 0.27 |
| Na₂HPO₄—7H₂O | 0.041 | 0.052 | 0.063 | 0.073 |
| H₂O | 18.8 | 23.7 | 28.7 | 33.6 |
| Xylitol | 20.0 | 20.0 | 20.0 | 20.0 |
| Gelatin | 6.0 | 6.0 | 6.0 | 6.0 |
| Oil Phase | | | | |
| Wecobee M | 55.0 | 50.0 | 45.0 | 40.0 |
| Homogenous? | No | No | Yes | Yes |

Weights were not recorded on the final articles of Comparative Examples C8 and C9 so the final weight percentages after water loss, e.g., in the useful articles cannot be calculated. Table 7 however, shows the final weights for Examples 11 and 12 to show the weight change in the articles.

TABLE 7
all amounts presented in weight percent (wt-%)

| | Comp. Ex. | Comp. Ex. | | |
|---|---|---|---|---|
| | C8 | C9 | Ex. 11 | Ex. 12 |
| Initial Lozenge | 8.01 | 7.7 | 4.07 | 6.59 |
| Weight (g) | | | | |
| Final Lozenge | 7.40 | 6.73 | 3.29 | 5.13 |
| Weight (g) | | | | |
| Water Phase | | | | |
| CaCl₂ | 0.0012 | 0.0013 | 0.0012 | 0.0015 |
| MgCl₂—6H₂O | 0.0012 | 0.0013 | 0.0012 | 0.0015 |
| KCl | 0.0025 | 0.0026 | 0.0024 | 0.0030 |
| KH₂PO₄ | 0.0025 | 0.0026 | 0.0024 | 0.0030 |
| NaCl | 0.098 | 0.10 | 0.098 | 0.12 |
| Na₂HPO₄—7H₂O | 0.026 | 0.028 | 0.026 | 0.033 |
| H₂O | 12.2 | 12.9 | 12.1 | 15.0 |
| Xylitol | 21.6 | 22.9 | 24.7 | 25.7 |
| Gelatin | 6.5 | 6.9 | 7.4 | 7.7 |
| Oil Phase | | | | |
| Wecobee M | 59.5 | 57.2 | 55.7 | 51.4 |
| Homogenous? | No | No | Yes | Yes |

The articles of Comparative Examples C10 and C11 showed examples of the separation of oil and water phases upon re-heating to 45° C. after homogenizing. In contrast, Examples 11 and 12 both stayed homogenous.

### Comparative Example C12 Using Hydroxypropyl Guar

The above-described method was used, except that the hydroxypropyl guar was first mixed with glycerin before being added to the water phase. The water and oil phases were mixed using the homogenizer as described above. Ingredients were added at the following weight percentages seen in Table 8

TABLE 8

| | Comp. Ex. |
|---|---|
| | C12 |
| Water Phase | |
| CaCl₂ | 0.0030 |
| MgCl₂—6H₂O | 0.0030 |
| KCl | 0.0060 |
| KH₂PO₄ | 0.0060 |
| NaCl | 0.24 |
| Na₂HPO₄—7H₂O | 0.064 |
| H₂O | 29.5 |
| Xylitol | 7.9 |
| Guar (Solvay Jaguar HP-8 COS) | 0.8 |
| Glycerin | 1.5 |
| Oil Phase | |
| Wecobee M | 54.4 |
| 10-1-S | 4.4 |
| 6-2-S | 1.1 |

The composition of Comparative Example C12 was poured into a silicone mold. The composition did not set at room temperature within 15 minutes. Even after the next day, the sample was still liquid inside.

Illustrative embodiments include:

An article including not greater than 60 wt-% of one or more plant based oil that is solid at 25° C. (room temperature), the weight percent based on the total weight of the article; and from 4 wt-% to 10 wt-% of gelatin, from 0.8 to 5 wt-% of agar, or a combination thereof, the weight percent based on the total weight of the article, wherein the article is solid set and homogeneous.

Articles according to any of the above embodiments, wherein the one or more oil that is solid at 25° C. is selected from hydrogenated vegetable oil.

Articles according to any of the above embodiments, wherein the one or more oil that is solid at 25° C. is present in an amount not less than 5 wt-% based on the total weight of the article.

Articles according to any of the above embodiments, wherein the one or more oil that is solid at 25° C. is present in an amount from 10 wt-% to 57 wt-% based on the total weight of the article.

Articles according to any of the above embodiments, wherein the one or more plant based oils are present in an amount from 15 wt-% to 56 wt-% based on the total weight of the article.

Articles according to any of the above embodiments, wherein the gelatin is present in an amount from 4 wt-% to 10 wt-% based on the total weight of the article.

Articles according to any of the above embodiments, wherein the gelatin is present in an amount from 5 wt-% to 9 wt-% based on the total weight of the article.

Articles according to any of the above embodiments, wherein the gelatin is present in an amount from 6 wt-% to 8 wt-% based on the total weight of the article.

Articles according to any of the above embodiments, wherein the agar is present in an amount from 1 wt-% to 4 wt-% based on the total weight of the article.

Articles according to any of the above embodiments, wherein the agar is present in an amount from 1.2 wt-% to 2 wt-% based on the total weight of the article.

Articles according to any of the above embodiments, wherein the agar is present in an amount from 1.2 wt-% to 2 wt-% based on the total weight of the article.

Articles according to any of the above embodiments further comprising water in an amount from 10 to 35 wt-% based on the total weight of the article

Articles according to any of the above embodiments further comprising water in an amount from 12 to 34 wt-% based on the weight of the final article.

Articles according to any of the above embodiments further comprising sweeteners, mineral salts, buffering components, flavorants, preservative agents, humectants, or combinations thereof.

Articles according to any of the above embodiments further comprising aloe vera, folic acid, hyaluronic acid, ceramides, glycine, arginine, betaines or oxygenated glycerol triesters, vitamin E, vitamin B12, EDTA, cetyl pyridinium chloride, chlorhexidine, other antiseptics, or combinations thereof.

Articles according to any of the above embodiments, wherein the article comprises from 1 wt-% to 30 wt-% of one or more sweetener based on the total weight of the article.

Articles according to any of the above embodiments, wherein the article is solid set.

Articles according to any of the above embodiments, wherein the article is homogeneous.

Articles according to any of the above embodiments, wherein the article is solid set and homogenous after being subjected to mixing, re-heating, cooling, or any combination thereof.

Articles according to any of the above embodiments, wherein the article can prevent, inhibit, disrupt, or any combination thereof the formation or maintenance of a biofilm in an area contacted with the article(s).

Articles according to any of the above embodiments, wherein the article can affect hydration loss in an area contacted by the article(s).

Articles according to any of the above embodiments, wherein the article can affect lubricity or lubriciousness of an area contacted by the article(s).

Methods of preventing, inhibiting, disrupting, or any combination thereof the formation or maintenance of a biofilm in an oral tissue, the method comprising: contacting an oral tissue with an article according to any of the above embodied articles.

Methods of affecting hydration loss in an oral tissue, the method comprising: contacting an oral tissue with an article according to any of the above embodied article.

Methods of affecting lubricity or lubriciousness in an oral tissue, the method comprising: contacting an oral tissue with any of the above embodied articles.

Methods of affecting the effects of xerostomia, dry mouth, or both, the method comprising: contacting an oral tissue with any of the above embodied article(s).

A composition for forming an article, the composition comprising: not greater than 50 wt-% of one or more plant based oil that is solid at 25° C. s, the weight percent based on the total weight of the composition; from 2 wt-% to 15 wt-% of gelatin, from 0.6 to 4.8 wt-% of agar, or a combination thereof, the weight percent based on the total weight of the composition; and from 20 wt-% to 60 wt-% water based on the total weight of the composition.

The composition according to any of the above embodied compositions, wherein the composition comprises from 5 wt-% to 48 wt-% of one or more plant based oils that is solid at 25° C. based on the total weight of the composition.

The composition according to any of the above embodied compositions, wherein the composition comprises from 10 wt-% to 45 wt-% of one or more plant based oils that is solid at 25° C. based on the total weight of the composition.

The composition according to any of the above embodied compositions, wherein the composition comprises from 15 wt-% to 40 wt-% of one or more plant based oils that is solid at 25° C. based on the total weight of the composition.

The composition according to any of the above embodied compositions, wherein the composition comprises from 4 wt-% to 10 wt-% gelatin based on the total weight of the composition.

The composition according to any of the above embodied compositions, wherein the composition comprises from 5 wt-% to 8 wt-% gelatin based on the total weight of the composition.

The composition according to any of the above embodied compositions, wherein the composition comprises from 1 wt-% to 3.8 wt-% agar based on the total weight of the composition.

The composition according to any of the above embodied compositions, wherein the composition comprises from 1.2 wt-% to 1.8 wt-% agar based on the total weight of the composition.

The composition according to any of the above embodied compositions, wherein the composition comprises from 25 wt-% to 57 wt-% water based on the total weight of the composition.

The composition according to any of the above embodied compositions further comprising sweeteners, mineral salts, buffering components, flavorants, preservative agents, humectants, or combinations thereof.

The composition according to any of the above embodied compositions further comprising aloe vera, folic acid, hyaluronic acid, ceramides, glycine, arginine, betaines or oxygenated glycerol triesters, vitamin E, vitamin B12, EDTA, cetyl pyridinium chloride, chlorhexidine, other antiseptics, or combinations thereof.

The composition according to any of the above embodied compositions, wherein the composition comprises from 10 wt-% to 30 wt-% of one or more sweetener based on the total weight of the composition

The composition according to any of the above embodied compositions, wherein the composition was formed by combining an oil based composition with a water based composition.

Thus embodiments of oral compositions and methods of use are disclosed. The implementations described above and other implementations are within the scope of the following claims. One skilled in the art will appreciate that the present disclosure can be practiced with embodiments other than those disclosed. The disclosed embodiments are presented for purposes of illustration and not limitation.

1. An article comprising:
one or more plant-based oil present in an amount not greater than 60 wt-%; and
one or more of:
gelatin present in amount from 4 wt-% to 10 wt-%, and
agar present in amount from 0.8 to 5 wt-%,
wherein the one or more plant-based oil is a solid at 25° C.,
wherein the article is solid set and homogeneous, and
wherein each wt-% is with respect to the weight of the article.

2. The article according to claim 1, wherein the one or more plant-based oil is a hydrogenated vegetable oil.

3. The article according to claim 1, wherein the one or more plant-based oil is present in an amount of at least 5 wt-% with respect to the weight of the article.

4. The article according to claim 1, wherein the one or more plant-based oil is present in an amount from 10 wt-% to 57 wt-% with respect to the weight of the article.

5. The article according to claim 1, wherein the gelatin is present in an amount from 4 wt-% to 10 wt-% with respect to the weight of the article.

6. The article according to claim 1, wherein the agar is present in an amount from 1 wt-% to 4 wt-% with respect to the weight of the article.

7. The article according to claim 1, further comprising water in an amount from 10 to 35 wt-% with respect to the weight of the article.

8. The article according to claim 1, further comprising sweeteners, mineral salts, buffering components, flavorants, preservative agents, humectants, or a combination thereof.

9. The article according to claim 1, further comprising aloe vera, folic acid, hyaluronic acid, ceramides, glycine, arginine, betaines or oxygenated glycerol triesters, vitamin E, vitamin B12, EDTA, cetyl pyridinium chloride, chlorhexidine, antiseptics, or a combination thereof.

10. The article according to claim 1, wherein the article has
one or more of the following uses:
one or more of prevents, inhibits, and disrupts, the formation or maintenance of a biofilm in an area contacted with the article;
reduces hydration loss in an area contacted by the article; and
increases lubricity or lubriciousness of an area contacted by the article.

11. A method of one or more of preventing, inhibiting, and disrupting the formation or maintenance of a biofilm in an oral tissue, the method comprising:
contacting an oral tissue with an article according to claim 1.

12. A method of reducing hydration loss in an oral tissue, the method comprising:
contacting an oral tissue with an article according to claim 1.

13. A method of increasing lubricity or lubriciousness in an oral tissue, the method comprising:
contacting an oral tissue with an article according to claim 1.

14. A method of reducing the effects of xerostomia, dry mouth, or both, the method comprising:
contacting an oral tissue with an article according to claim 1.

15. A composition for forming an article, the composition comprising:
one or more plant-based oil present in amount not greater than 50 wt-%;
one or more of:
gelation present in amount from 2 wt-% to 15 wt-%, and
agar present in amount from 0.6 to 4.8 wt-%; and
water present in amount from 20 wt-% to 60 wt-%,
wherein the one or more plant-based oil is solid at 25° C., and
wherein wt-% is with respect to the weight of the composition.
